(19) United States
(12) Patent Application Publication
(10) Pub. No.: US 2024/0390108 A1
(43) Pub. Date: Nov. 28, 2024

(54) SELF-RETAINING SURGICAL FLAP RETRACTOR/HOLDER FOR DENTAL SURGICAL PROCEDURES

(71) Applicant: KING FAISAL UNIVERSITY, AL-AHSA (SA)

(72) Inventor: NAZARGI MAHABOB, AL-AHSA (SA)

(21) Appl. No.: 18/200,479

(22) Filed: May 22, 2023

(51) Int. Cl. A61C 3/00; A61B 90/30; A61C 17/08

(52) U.S. Cl. CPC A61C 3/00; A61B 90/30; A61C 17/08; A61B 2090/309

(57) ABSTRACT

A self-retaining surgical flap retractor and method of using the same for the surgical extraction of impacted third mandibular molars of a patient. Once optimally positioned, the self-retaining surgical retractor pushes against the mucoperiosteal flaps keeping them away from the surgical site. The system and method also provide integrated functionalities of suction and illumination within the self-retaining surgical retractor.

### BACKGROUND

### 1. Field

The present disclosure relates to self-retaining retractors used in oral or dental surgical procedures.

### 2. Description of the Related Art

The importance of surgical retractors is well known in surgical procedures. Surgical retractors are very important because they determine the exposure of the operative field. Surgical retractors are available in many sizes, shapes, and styles. Surgical retractors can be hand-held or self-retaining dependent upon the site requiring the operation. Retractors used in oral or dental surgical procedures are primarily involved in moving mucoperiosteal flaps away and holding them away from a surgical site to improve both visibility and access to the surgical site. They aid in providing an ingress for removing foreign bodies or teeth from the oral cavity.

Self-retaining retractors permit a medical practitioner a hands-free operation during a surgical operation. For oral surgical procedures, there are several types of retractors being used. Common self-retaining retractors used in surgery include: Meyerding, Markham-Meyerding, Gelpi, Cerebellar Jansen, Weitlander and Williams retractors. These are all based on a solid, stainless-steel frame with a self-locking, retaining mechanism. Surgical retractors are typically non-hollow components made from materials that can be placed in an autoclave to be sterilized and so they may be reused for multiple, successive oral surgical procedures. There are a few surgical retractors that have been designed with integrated suction and external light sources mounted upon the tool to allow the oral surgeon to perform an evacuation process at the same time they perform the surgical procedure. Although there are prior art devices that combine suction and an external light source, none have adequately been integrated into standard self-retaining retraction devices. Advancement in surgical techniques and devices have included several devices for suction and retraction as well as “minimally invasive techniques.” However, these devices and techniques, while potentially reducing tissue trauma, lend themselves to increased operating time and thus, increased exposure to anesthesia and infection increases. However, there are no specific self-retaining retractors for the surgical extraction of impacted third mandibular molars.

What is needed is a self-retaining retractor for the surgical extraction of impacted third mandibular molars which integrates an external light source and suction, thereby freeing the operator to perform the intricate tasks of surgery unencumbered by the need to rely upon additional tools and instruments during the operation.

### SUMMARY

The present subject matter is directed towards a self-retaining surgical flap retractor and method of using the same for the surgical extraction of impacted third mandibular molars.

In a first aspect, the present subject matter relates to a self-retaining surgical flap retractor for surgical extraction of impacted third mandibular molars of a patient, comprising: a first arm having a first handle at a first end of the first arm and having a second end of the first arm; a second arm having a second handle at a first end of the second arm and having a second end of the second arm; a fastener connecting the first arm and the second arm together; a spring or coil situated between the first and the second arm and between the fastener and the respective first ends of the first arm and the second arm that provides an inverse function when the first handle and the second handle are squeezed together thereby opening apart the respective second ends of the first arm and the second arm, wherein a broad, round-edged, and curved flap retractor plate situated at the second end of said second arm is configured to push against the patient's mucoperiosteal flap and keep the mucoperiosteal flap separated from a surgical site of the patient when the first handle and the second handle are squeezed together; a LED light on top of the second end of the second arm for providing illumination to the surgical site; a suction between the respective second ends of the first arm and the second arm having a disposable suction tip at a first end of the suction for providing suction to the surgical site; and a lock formed by squeezing said first handle and said second handle together wherein by said squeezing, the self-retaining surgical flap retractor is configured to maintain a fixed position and alignment within an oral cavity of said patient.

In another aspect, the present subject matter relates to a method for moving mucoperiosteal flaps holding them away from a surgical site during surgical extraction of impacted third mandibular molars of a patient, comprising: providing a self-retaining surgical flap retractor comprising: a first arm having a first handle at a first end of the first arm and having a second end of the first arm; a second arm having a second handle at a first end of the second arm and having a second end of the second arm; a fastener connecting the first arm and the second arm together; a spring or coil situated between the first and the second arm and between the fastener and the respective first ends of the first arm and the second arm that provides an inverse function when the first handle and the second handle are squeezed together, wherein a broad, round-edged, and curved flap retractor plate is situated at the second end of said second arm; a LED light on top of the second end of the second arm for providing illumination to the surgical site; a suction between the respective second ends of the first arm and the second arm having a disposable suction tip at a first end of the suction for providing suction to the surgical site; and a lock formed by squeezing said first handle and said second handle together; placing the respective second ends of the first arm and the second arm inside a mouth of the patient; squeezing the respective first ends of the first handle and the second handle together, thereby opening apart the respective second ends of the first arm and the second arm such that the broad, round-edged, and curved flap retractor plate pushes against the patient's mucoperiosteal flap and keeps the mucoperiosteal flap separated from the surgical site of the patient and a waved surface at the second end of the first arm contacting a tooth of said patient; and locking the first handle and the second handle together such that the self-retaining surgical flap retractor maintains a fixed position and alignment within an oral cavity of said patient.

These and other features of the present subject matter will become readily apparent upon further review of the following specification.

### BRIEF DESCRIPTION OF THE DRAWINGS

FIG. 1 is a diagram of the self-retaining surgical flap retractor/holder for dental surgical procedure.

Similar reference characters denote corresponding features consistently throughout the attached drawings.

### DETAILED DESCRIPTION OF EMBODIMENTS

The following detailed description is merely exemplary in nature and is not intended to limit the described embodiments or the application and uses of the described embodiments. Any implementation described herein with the words “exemplary” or “illustrative” is not necessarily construed as preferred or advantageous over other implementations.

All of the implementations described below are exemplary implementations provided to enable persons skilled in the art to make or use the embodiments of the disclosure and are not intended to limit the scope of the disclosure, which is defined by the claims. For the purposes of the description herein, there is no intention to be bound by any expressed or implied theory presented in the preceding technical field, background, brief summary or the following detailed description.

It is also to be understood that the specific devices and processes illustrated in the attached drawings, and described in the following specification, are simply exemplary embodiments of the present subject matter defined in the appended claims. Hence, specific dimensions and other physical characteristics relating to the embodiments disclosed therein are not to be considered as limiting, unless the claims expressly state otherwise.

The present subject matter is directed towards a self-retaining surgical flap retractor and method of using the same for the surgical extraction of impacted third mandibular molars.

In a first aspect, the present subject matter relates to a self-retaining surgical flap retractor for surgical extraction of impacted third mandibular molars of a patient, comprising: a first arm having a first handle at a first end of the first arm and having a second end of the first arm; a second arm having a second handle at a first end of the second arm and having a second end of the second arm; a fastener connecting the first arm and the second arm together; a spring or coil situated between the first and the second arm and between the fastener and the respective first ends of the first arm and the second arm that provides an inverse function when the first handle and the second handle are squeezed together thereby opening apart the respective second ends of the first arm and the second arm, wherein a broad, round-edged, and curved flap retractor plate situated at the second end of said second arm is configured to push against the patient's mucoperiosteal flap and keep the mucoperiosteal flap separated from a surgical site of the patient when the first handle and the second handle are squeezed together; a LED light on top of the second end of the second arm for providing illumination to the surgical site; a suction between the respective second ends of the first arm and the second arm having a disposable suction tip at a first end of the suction for providing suction to the surgical site; and a lock formed by squeezing said first handle and said second handle together wherein by said squeezing, the self-retaining surgical flap retractor is configured to maintain a fixed position and alignment within an oral cavity of said patient.

In one embodiment, the first arm can be a resting arm which has a waved surface at the second end of the first arm, said waved surface configured for contacting a tooth of said patient. In this regard, the waved surface can be configured for contacting a buccal side of the tooth of the patient.

In another embodiment, the lock used in the present retractor is a serrated lock.

In a further embodiment, the LED light used in the present retractor is detachable. Further, the LED light can be situated on a flat surface of the broad, round-edged, and curved flap retractor plate. In this regard, a diameter of the broad, round-edged, and curved flap retractor plate can be about 2.5 to about 3 cm.

In one embodiment, the self-retaining surgical retractor can be made from stainless steel.

In another aspect, a second end of the suction used in the present retractor can be attached to the first arm or the second arm in a groove in the first arm situated between the fastener and the respective second ends of the first arm or the second arm.

In an embodiment, the self-retaining surgical retractor can be configured for application to a right side or a left side of the patient's mouth.

In another embodiment, at rest, the first arm and the second arm are approximately parallel to one another.

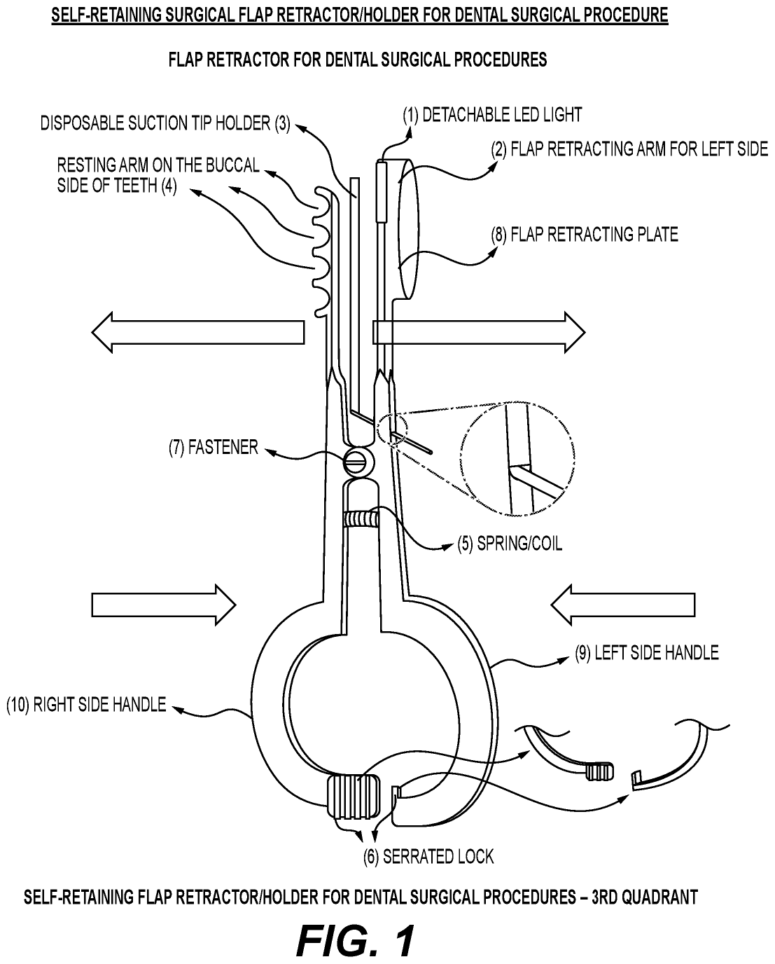

In the embodiment of FIG. 1, a self-retaining surgical retractor includes a first arm (4), a second arm (2) connected to a first arm by a fastener (7), a first handle (10) attached to a first end of the first arm (4), a second handle (9) attached to a first end of the second arm (2), a waved surface at a second end of the first arm (4), and a broad, round edged and curved flap retractor plate (8) attached to a second end of the second arm (2). The broad, round edged, and curved flap retractor plate in this embodiment is 2.5-3 cm in diameter. A spring or coil (5) is situated between the first and second arms (4, 2) and provides an inverse function to the first and second arms when the first and second handles (10, 9) are squeezed together. A serrated snap-lock (6) when closed connects the first handle (10) to the second handle (9) in a locked configuration based upon a sliding action of the left side and across the serrations of the right side handle. A detachable LED light (1) is mounted on the curved flap retractor plate (8) and provides illumination to the surgical site. Also shown in FIG. 1, is a disposable suction tip holder (3) for holding an attached suction tube to the instrument such that suction is applied when desired.

In operation, the self-retaining surgical retractor, when deployed by the medical practitioner will be initially inserted into the oral cavity in a closed configuration. When proximal to the surgical site, the medical practitioner squeezes the right side handle (10) and the left side handle (9) together, a movement as shown by the inward arrows, the distal arms will expand out as shown by the outward arrows such that the flap retracting arm (2) on the left side of the self-retaining surgical retractor is pushed against the patient's mucoperiosteal flap and the broad round edged and curved flap retractor plate (8) keeps the mucoperiosteal flap separated from the surgical site. Both arms (2, 4) have small grooves providing an anchor point to affix the disposable suction tip holder (3). As shown in FIG. 1, the disposal suction tip holder (3) is affixed to the groove of the flap retracting arm (2). Once the self-retaining retractor is positioned as desired and the right side resting arm (4) is situated on the buccal side of the teeth for support, the serrated lock (6) is slidingly closed so that the self-retaining retractor fixes the position and alignment as desired by the medical practitioner when performing a surgical extraction of impacted mandibular third molars.

Additionally, the LED light (1) is provided on the flat side of the broad round edged and curved flap retractor plate (8) and is turned on to provide illumination to the surgical site. The LED light (1) is detachable from the flat side of the flap retracting plate (8). The location of the attached LED light allows the medical practitioner to properly view the surgical area without the need for additional tools. Further, the location of the LED light (1) on the flat side of the flap retracting plate (8) eliminates the need for an assistant to provide a separate lighting device, thereby reducing the time needed to perform the procedure.

The location of the disposable suction tip holder (3) allows for the medical practitioner to properly evacuate smoke, debris, liquids, and other objects from the surgical area without the need for additional tools. Further the location of the disposable suction tip holder (3) also eliminates the need for an assistant to provide a separate suction device, thereby also reducing time needed to perform the procedure.

According to the exemplary embodiment of FIG. 1, the elements of the self-retaining surgical retractor are formed of stainless steel. However, the retractor may be formed of any material known in the art which is suitable for use in surgical procedures. Further, materials that are suitable for use in surgical procedures can be cleaned by autoclaving or other similar techniques are preferable. In addition, the suction tube may be made of any material suitable for use in surgical procedures, including but not limited to polymers used for surgical techniques.

Accordingly, the present subject matter relates to a method for moving mucoperiosteal flaps holding them away from a surgical site during surgical extraction of impacted third mandibular molars of a patient, comprising: providing a self-retaining surgical flap retractor comprising: a first arm having a first handle at a first end of the first arm and having a second end of the first arm; a second arm having a second handle at a first end of the second arm and having a second end of the second arm; a fastener connecting the first arm and the second arm together; a spring or coil situated between the first and the second arm and between the fastener and the respective first ends of the first arm and the second arm that provides an inverse function when the first handle and the second handle are squeezed together, wherein a broad, round-edged, and curved flap retractor plate is situated at the second end of said second arm; a LED light on top of the second end of the second arm for providing illumination to the surgical site; a suction between the respective second ends of the first arm and the second arm having a disposable suction tip at a first end of the suction for providing suction to the surgical site; and a lock formed by squeezing said first handle and said second handle together; placing the respective second ends of the first arm and the second arm inside a mouth of the patient; squeezing the respective first ends of the first handle and the second handle together, thereby opening apart the respective second ends of the first arm and the second arm such that the broad, round-edged, and curved flap retractor plate pushes against the patient's mucoperiosteal flap and keeps the mucoperiosteal flap separated from the surgical site of the patient and a waved surface at the second end of the first arm contacting a tooth of said patient; and locking the first handle and the second handle together such that the self-retaining surgical flap retractor maintains a fixed position and alignment within an oral cavity of said patient.

In an embodiment of the present methods, the self-retaining surgical flap retractor can independently maintain the fixed position and alignment within the oral cavity of said patient, permitting hands-free keeping of the mucoperiosteal flap separated from the surgical site of the patient.

In another embodiment of the present methods, said first arm is a resting arm which has a waved surface at the second end of the first arm and said waved surface contacts a tooth of said patient. In this regard, the waved surface can contact a buccal side of the tooth of the patient. Further, the lock can be a serrated lock and the self-retaining surgical retractor can be made from stainless steel.

In one aspect, a second end of the suction can be attached to the first arm or the second arm in a groove in the first arm situated between the fastener and the respective second ends of the first arm or the second arm. In another aspect, the self-retaining surgical retractor can be applied to a right side or a left side of the patient's mouth. Further, at rest, the first arm and the second arm are approximately parallel to one another.

It is to be understood that the method and device for using a self-retaining surgical flap retractor for the surgical extraction of impacted third mandibular molars is not limited to the specific embodiments described above but encompasses any and all embodiments within the scope of the generic language of the following claims enabled by the embodiments described herein, or otherwise shown in the drawings or described above in terms sufficient to enable one of ordinary skill in the art to make and use the claimed subject matter.

1. A self-retaining surgical flap retractor for surgical extraction of impacted third mandibular molars of a patient, comprising:
a first arm having a first handle at a first end of the first arm and having a second end of the first arm;
a second arm having a second handle at a first end of the second arm and having a second end of the second arm;
a fastener connecting the first arm and the second arm together;
a spring or coil situated between the first and the second arm and between the fastener and the respective first ends of the first arm and the second arm that, when the first handle and the second handle are squeezed together, opens apart the respective second ends of the first arm and the second arm, wherein a broad, round-edged, and curved flap retractor plate situated at the second end of said second arm is configured to push against a patient's mucoperiosteal flap and keep the mucoperiosteal flap separated from a surgical site of the patient when the first handle and the second handle are squeezed together;
a LED light on top of the second end of the second arm for providing illumination to the surgical site;
a suction port between the second ends of the first arm and the second arm having a disposable suction tip at a first end of the suction port for providing suction to the surgical site; and
a lock located between said first end of said first handle on a first curved portion of said first handle and said first end of said second handle on a first curved portion of said second handle wherein by squeezing the first curved portion of said first handle together with the first curved portion of said second handle, the self-retaining surgical flap retractor is configured to maintain a fixed position and alignment within an oral cavity of said patient,
wherein the LED light is detachable.

2. The self-retaining surgical flap retractor as recited in claim 1, wherein said first arm is a resting arm which has a waved surface at the second end of the first arm, said waved surface configured for contacting a tooth of said patient.

3. The self-retaining surgical flap retractor as recited in claim 2, wherein said waved surface is configured for contacting a buccal side of the tooth of the patient.

4. The self-retaining surgical flap retractor as recited in claim 1, wherein the lock is a serrated lock.

5. (canceled)

6. The self-retaining surgical flap retractor as recited in claim 1, wherein the LED light is situated on a flat surface of the broad, round-edged, and curved flap retractor plate.

7. The self-retaining surgical flap retractor as recited in claim 1, wherein a diameter of the broad, round-edged, and curved flap retractor plate is about 2.5 to about 3 cm.

8. The self-retaining surgical flap retractor as recited in claim 1, wherein the self-retaining surgical retractor is made from stainless steel.

9. The self-retaining surgical flap retractor as recited in claim 1, wherein a second end of the suction port is attached in a groove in the first arm situated between the fastener and the second ends of the first arm or the second arm.

10. The self-retaining surgical flap retractor as recited in claim 1, wherein the self-retaining surgical retractor is configured for application to a right side or a left side of a patient's mouth.

11. The self-retaining surgical flap retractor as recited in claim 1, wherein, at rest, the first arm and the second arm are approximately parallel to one another.

12. A method for moving mucoperiosteal flaps by holding them away from a surgical site during surgical extraction of impacted third mandibular molars of a patient, comprising:
providing a self-retaining surgical flap retractor comprising:
a first arm having a first handle at a first end of the first arm and having a second end of the first arm;
a second arm having a second handle at a first end of the second arm and having a second end of the second arm;
a fastener connecting the first arm and the second arm together;
a spring or coil situated between the first and the second arm and between the fastener and the first ends of the first arm and the second arm, wherein a broad, round-edged, and curved flap retractor plate is situated at the second end of said second arm;
a LED light on top of the second end of the second arm for providing illumination to the surgical site;
a suction port between the respective second ends of the first arm and the second arm having a disposable suction tip at a first end of the suction port for providing suction to the surgical site; and
a lock located between said first handle and said second handle together;
placing the second ends of the first arm and the second arm inside a mouth of the patient;
squeezing the first ends of the first handle and the second handle together, thereby opening apart the second ends of the first arm and the second arm such that the broad, round-edged, and curved flap retractor plate pushes against the patient's mucoperiosteal flap and keeps the mucoperiosteal flap separated from the surgical site of the patient and a waved surface at the second end of the first arm contacting a tooth of said patient; and
locking a first curved portion of the first handle having the first end of the first handle and a first curved portion of the second handle having the first end of the second handle together such that the self-retaining surgical flap retractor maintains a fixed position and alignment within an oral cavity of said patient,
wherein the LED light is detachable.

13. The method of claim 12, wherein the self-retaining surgical flap retractor independently maintains the fixed position and alignment within the oral cavity of said patient, permitting hands-free keeping of the mucoperiosteal flap separated from the surgical site of the patient.

14. The method of claim 12, wherein said first arm is a resting arm which has a waved surface at the second end of the first arm and said waved surface contacts a tooth of said patient.

15. The method of claim 14, wherein said waved surface contacts a buccal side of the tooth of the patient.

16. The method of claim 12, wherein the lock is a serrated lock.

17. The method of claim 12, wherein the self-retaining surgical retractor is made from stainless steel.

18. The method of claim 12, wherein a second end of the suction port is attached in a groove in the first arm situated between the fastener and the second ends of the first arm or the second arm.

19. The method of claim 12, wherein the self-retaining surgical retractor is applied to a right side or a left side of the patient's mouth.

20. The method of claim 12, wherein, at rest, the first arm and the second arm are approximately parallel to one another.
